(19) United States
(12) Patent Application Publication
(10) Pub. No.: US 2019/0015541 A1
(43) Pub. Date: Jan. 17, 2019

(54) VIRUS SHIELD

(71) Applicant: Honeywell International Inc., Morris Plains, NJ (US)

(72) Inventor: Andrzej Peczalski, Edina, MN (US)

(21) Appl. No.: 16/033,938

(22) Filed: Jul. 12, 2018

Related U.S. Application Data
(60) Provisional application No. 62/531,529, filed on Jul. 12, 2017.

(51) Int. Cl. A61L 9/20; A61M 16/06

(52) U.S. Cl. CPC A61L 9/20; A61L 2209/111; A61L 2209/12; A61M 16/0616

(57) ABSTRACT

Disclosed embodiments relate to a virus shield, which for example might be used with a mask worn to protect a user. The virus shield mask might include a container having a UV light source configured to effectively eliminate viruses, thereby allowing for effective virus protection for the user without traditional bulky virus filters. Disclosed embodiments may also include safety features to prevent unwanted or harmful UV exposure.

### CROSS-REFERENCE TO RELATED APPLICATIONS

This application is a non-provisional application claiming priority under 35 USC 119 to U.S. Provisional Patent Application 62/531,529 filed Jul. 12, 2017 by Andrzej Peczalski and entitled “Virus Shield”, which is hereby incorporated herein by reference for all purposes as if reproduced in its entirety.

### STATEMENT REGARDING FEDERALLY SPONSORED RESEARCH OR DEVELOPMENT

Not applicable.

### REFERENCE TO A MICROFICHE APPENDIX

Not applicable.

### FIELD

Disclosed embodiments relate to virus protection for a user, and specifically to masks configured to safely protect a user from viruses without significantly impacting mobility and/or comfort.

### BACKGROUND

There are many persons who, on a daily basis, might be exposed to airborne viruses which could pose a health risk. This may be particularly true in some fields of employment, but can even be true in ordinary everyday life (especially in circumstances such as aboard an airplane in which many people are packed together tightly in a confined area). It might be useful to provide a filtering mask for such persons, to reduce the spread of such airborne viruses. Unfortunately, the filtering of viruses is very difficult because they are smaller than 0.1 micron in size. Typical filtering masks may be made of cloth, and therefore cannot effectively trap the very small viruses. The type of masks which might provide effective filters capable of trapping viruses would need to be very bulky and/or would restrict airflow significantly (e.g. making breathing difficult/laborious), and therefore their adoption would be difficult due to aesthetics, practicality, and comfort.

### SUMMARY

Disclosed embodiments may relate to improved virus protection masks, as described herein. In an embodiment, a face mask might comprising a container configured to expose breathable air to ultraviolet radiation (e.g. before allowing entry of air into the interior of the mask), the container comprising: at least one inlet allowing air flow into the container; at least one light source configured to produce ultraviolet radiation, wherein the ultraviolet radiation is configured to kill viruses and/or other pathogens within the air flow; a plurality of internal surfaces configured to reflect the ultraviolet radiation; and at least one outlet allowing air flow out of the container toward a user's nose and/or mouth (e.g. the interior of the mask).

In an embodiment, a container (which might be used with a mask, for example configured so that airflow into the mask must pass through the container) might be configured to expose breathable air to ultraviolet radiation, with the container for example comprising: at least one inlet allowing air flow into the container; at least one light source configured to produce ultraviolet radiation, wherein the ultraviolet radiation is configured to kill viruses within the air flow; a plurality of internal surfaces configured to reflect the ultraviolet radiation; and at least one outlet allowing air flow out of the container toward a user's nose and/or mouth.

In an embodiment, a method (for preventing viruses from reaching a user's mouth and/or nose) might comprise: directing breathable air into a container via one or more inlets; emitting ultraviolet radiation within the container; (optionally, reflecting the ultraviolet radiation by the inner surfaces of the container); killing viruses contained in the air within the container; and directing the air out of the container via one or more outlets toward the user's mouth and/or nose (e.g. located within an interior of a mask).

### BRIEF DESCRIPTION OF DRAWINGS

For a more complete understanding of the present disclosure, reference is now made to the following brief description, taken in connection with the accompanying drawings and detailed description, wherein like reference numerals represent like parts.

FIG. 1 illustrates via a perspective view schematic an exemplary container (shown transparent so the inner-workings may be observed) configured to effectively reduce the risk of viruses using UV light;

FIG. 2 illustrates via a side view (shown transparent so the inner-workings may be observed) a similar exemplary container with shield elements over the inlets and outlet (e.g. configured to prevent UV light from exiting the container); and

FIG. 3 illustrates an enlarged perspective view of a shielded inlet on FIG. 2.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number (for example, +/−10%), as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Embodiments of the disclosure include systems and methods for protecting a user from exposure to viruses. A user may wear a mask or other breathing filter to prevent viruses from entering their body via the nose and/or mouth. Airborne viral diseases may be easily transmitted, particularly in crowded environments, like buses, airplanes, subways, and other commute environments. Some work environments expose workers to interactions with other people and their viruses, such as shop clerk, bank teller, doctor, nurse, and other service jobs. It is desirable to remove the threat of viruses before they enter nose and mouth. However, the filtering of viruses is very difficult because they are smaller than 0.1 micron in size. Typical masks may be made of cloth, and therefore cannot effectively trap the very small viruses. The masks with effective filters capable of trapping viruses would typically be very bulky and/or would have a large pressure drop (e.g. it would be very difficult/laborious for a user to breath while wearing the filter mask), and therefore their adoption would be difficult due to aesthetics, practical functionality, and/or comfort.

The embodiments disclosed herein may allow for a small mask that imposes little restriction on the user's breathing. The weight of the mask could also be low if the batteries and/or electronics are worn on the body with a thin wire running from the chin to a breast pocket, pendant, hat, or other similar attachment location (e.g. the weight of the mask itself could be reduced by having the batteries and/or electronics located elsewhere, off of the mask). A small, low-weight mask capable of filtering/killing viruses could be useful for commercial workers, medical workers, as well as everyday consumers who spend time in crowded environments (especially if this could be safely accomplished without the need for a high pressure drop across the mask (which might make breathing laborious)).

The embodiments of a mask disclosed herein employ ultraviolet (e.g. band C) light emitting diodes (e.g. UVC LED) for killing the viruses before reaching the user's mouth and/or nose. Other bands and wavelengths of the UV radiation could also be employed for killing viruses. However, Applicant has found that band C has the highest efficiency and therefore would require a smaller number of LEDs and possibly a smaller power source (or battery). Typically, a dose of UVC energy exceeding 11 j/m² would effectively kill any viruses that contact the UVC radiation in a mask breathing context (e.g. for the amount of time that air filtering through a disclosed mask with UV container would contact the UVC radiation)(although higher kill rates might require UVC dose of at least 33 j/m² or at least 100 j/m² in some instances, for example to further reduce virus survival rate). The volume of breathable air that needs to be exposed to the UVC radiation could be calculated from the size of the tidal breath and average breathing rate (e.g. of either a specific user (e.g. customized) or of an average/standard user (e.g. with some safety factor to ensure that the mask would be effective even in more unusual circumstances)). Additionally, the volume may include a design margin to work when a person breathes deeply or more frequently. In some embodiments of the mask, the exposed volume (e.g. the volume within the container and/or exposed to the UV light) may be approximately 125 cm³ (or alternatively from approximately 80 cm³ to approximately 200 cm³). This exposed volume may be enclosed in a container having reflective inner surfaces (e.g., a container that is metalized inside) configured to provide UV internal reflection. Typically, the mask would be configured so that airflow into the interior of the mask (e.g. where the user breathes) must pass through the container. Disclosed embodiments typically are configured to eliminate viruses (e.g. 90-99%) without excessive pressure drop (e.g. of the sort that might make it difficult for a user to breathe effectively while undertaking standard activities). Some embodiments of the disclosure may include a mask configured to kill and/or filter out other air or aerosol borne pathogens, such as bacteria, fungi spores, prions, etc. (for example using a filter and/or a fungicide, antibacterial, etc.).

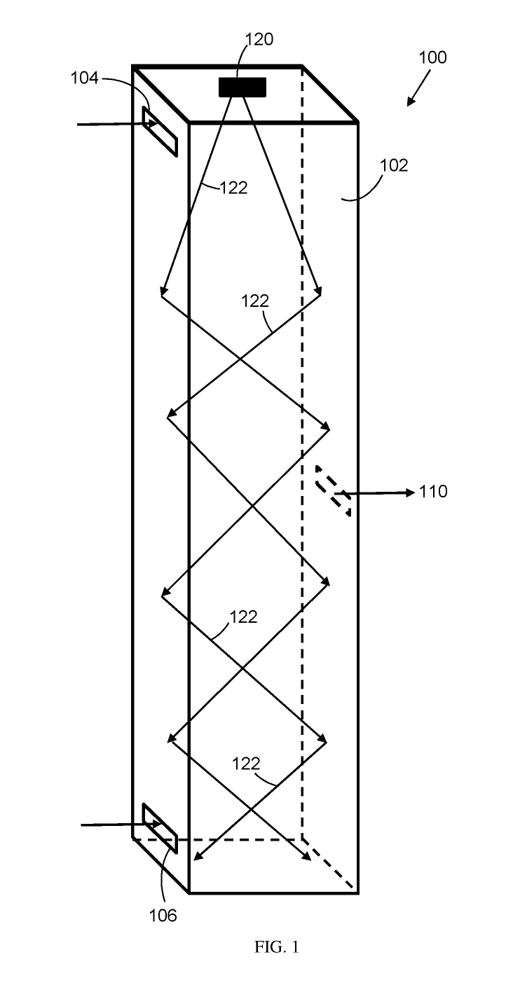

Referring to FIG. 1, an exemplary container 100 is shown, where the container 100 may be incorporated into, or formed into, a mask that may be worn by a user. The container 100 may comprise at least one inlet 104 and 106 and at least one outlet 110, where air may be drawn into the container 100 via the inlet(s) 104 and 106, and the air may be directed toward a user for breathing via the outlet 110. The container 100 may comprise a light source 120, which may comprise a UVC LED as described above, where the light source 120 may produce radiation 122, and the radiation 122 may be reflected multiple times within the volume of the container 100. The radiation 122 may interact with air flowing between the inlets 104 and 106 and the outlet 110, and may kill any viruses carried within the air flow.

For illustration purposes, the shape of the container 100 shown in FIG. 1 is a simple rectangular prism. However, the shape of the container 100 could be any three-dimensional volume comprising at least one inlet 104 and 106 and at least one outlet 110. As an example, the shape of the container could be ergonomically fit to a user's face (e.g. following contours similar to at least a portion of the corresponding mask). The shape of the container may be curved, rounded, etc. to better fit against a user's face. However, the internal surfaces 102 of the container 100 may comprise an optical design so the bouncing radiation 122 will reach the total volume of the container 100 with proper intensity. In other words, the internal surfaces 102 may be configured to provide UV reflection and/or may be configured with respect to the light source(s) 120 so that the (exposed) air volume within the container (e.g. between an inlet and the outlet) receives approximately uniform radiation, for example exceeding 11 j/m², 33 j/m², or 100 j/m² (or from 11-33 j/m², 33-100 j/m², or 11-100 j/m²). In some embodiments, the container 100 may comprise two or more light sources 120 located in different positions within the container 100 to improve the uniformity and intensity of the exposure of the airflow to the radiation 122. The air flow through the container 100 may be designed to be approximately symmetric, so the air flow has equal chance to be completely irradiated before exiting the container to the mouth and/or nose of a user. The inner curvature of the mask may be further optimized to increase the number of reflections of the UV light and therefore maximize the number of times that the air flow passing through the container 100 is exposed to the radiation 122.

The internal surfaces 102 of the container 100 may be made reflective, for example by being metalized, for efficient UV reflection. The components of the container 100 (e.g. the light source and/or the light detector and/or fan) may be powered by one or more batteries, and the batteries and/or electronics may be positioned on the container 100 (or elsewhere) to improve balance of the mask (e.g. with the weight distributed over several locations and/or symmetrically/evenly distributed about the vertical and/or horizontal centerline (e.g. balance point) of the mask), or may be tethered to the container 100 to minimize mask weight.

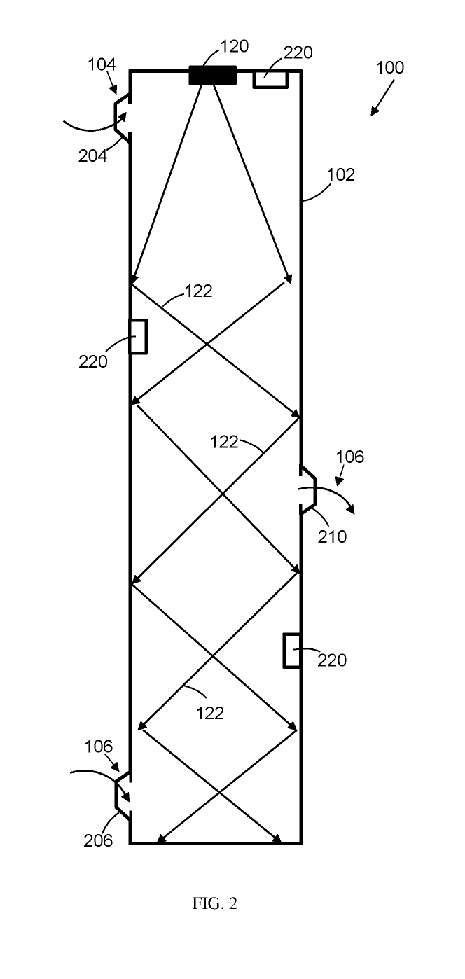

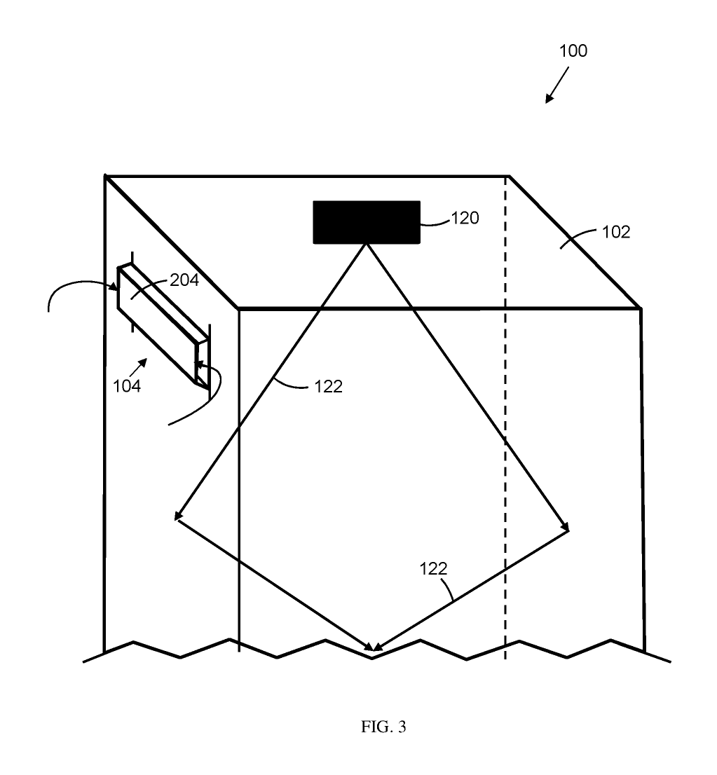

Referring to FIG. 2, the inlet(s) 104 and 106 and outlet(s) 110 of the container 100 may comprise one or more shields 204, 206, 210 configured to prevent stray radiation 122 from escaping the container 100, as the radiation 122 may be harmful to the user and/or to passers-by (e.g. others in proximity to the user). The shields 204, 206, 210 may comprise covers configured to allow airflow around the cover and into the container 100 (e.g. at a rate sufficient for user breathing, e.g. to provide air without significant pressure drop), and also configured to substantially prevent escape of any UV radiation out of the container 100 through inlet(s) or outlet(s) (e.g. by reflecting and/or absorbing any radiation 122 exiting the container and thereby encountering the shields). For example, a shield may be attached to the exterior of the container housing over the corresponding inlet/outlet. In some embodiments, the shield also may be configured to prevent outside light from entering the container and/or reaching the light sensor (as discussed below). FIG. 3 shows an enlarged view of an exemplary shield.

Additionally, the container 100 may comprise at least one safety light sensor 220 mounted inside the container 100. The safety light sensor 220 may be configured to detect if daylight (e.g. visible light and/or any light other than the UV light of the light source 120 of the container) has entered the interior of the container 100, thereby indicating a possible opening which could provide exposure to the radiation 122 (either to the user or to others in proximity to the user), and may be configured to deactivate the one or more light sources 120 when such light is detected. In some embodiments, light sensor 220 would be configured to only detect visible light and/or to not detect UV light (or perhaps not to detect UVC light), such that upon detecting any light, the light sensor 220 would automatically deactivate the light source 120 (e.g. by sending a signal to either a processor (which would then deactivate the light source) or to the light source).

Referring to FIG. 3, a detailed view of an exemplary shield 204 is shown, where the shield 204 may be positioned over an inlet 104 to the container 100 (e.g. on the exterior of the container in proximity to the corresponding inlet). The shield 204 may be similar to the shields 206 and 210 shown in FIG. 2. For example, the shield may have an outer panel spanning the corresponding inlet/outlet and two or more attachment panels (here shown as a top and a bottom panel) rigidly attaching the outer panel to the body/housing of the container in proximity to (e.g. encompassing) the corresponding inlet/outlet. In some embodiments, one or more sides of the shield may be open and/or may comprise openings to allow airflow therethrough, while in other such embodiments the side(s) may include an angled flap in conjunction with an opening configured (e.g. angled, shaped, and sized) to block UV light from exiting the corresponding side (e.g. opening) while allowing airflow into the shield (and thereby into the container). In some embodiments, at least the portion of the outer panel in proximity to the angled flap could be configured to absorb rather than reflect UV light (e.g. in order to ensure that UV light is not inadvertently reflected out of the shield past the angled flap) and/or the angled flap outer surface (e.g. facing away from the container exposed volume cavity) could be configured to absorb UV light (perhaps with the angled flap inner surface configured to reflect UV light back into the container in some embodiments), and/or in some embodiments, the remaining portion of the outer panel could be reflective of UV light (to return such UV light back into the inner hollow chamber of the container to further interact with the exposed volume). Similar shields could be used on the outlet(s) as well.

In some embodiments, the air flow into and out of the container may be controlled by changes in pressure caused by a user's breathing (e.g. the mask would be configured without significant pressure drop, so that the user's breathing would be sufficient to comfortably move air through the chamber and into the interior of the mask). In some embodiments, the container 100 may be attached to a fan or other similar air flow device configured to produce air flow through the container 100. In some embodiments, the container 100 may be attached to a mouth piece, mask shell, face strap, ear strap, neck strap, or any other mask element configured to convey air flow from the container to a user's mouth and/or nose.

As a specific example of the radiation that may be used to kill viruses with the container 100, Applicant has found that to achieve a 0.9 kill ratio of influenza virus at 75% humidity requires a UV (kill efficacy at approximately 254 nm-278 nm) dose of approximately 11 J/m² (see for example James J. McDevitt, Stephen N. Rudnick, and Lewis J. Radonovich Applied and Environmental Microbiology, March 2012 p. 1666-1669, incorporated by reference herein to the extent that it does not conflict with the specific principles set forth herein). Similarly, a 0.999 influenza kill ratio typically would require a UV dose of approximately 33 J/m². Thus, the container would typically be configured to provide UVC of at least 11 j/m², 33 j/m², or 100 j/m² (or from 11-33 j/m², 33-100 j/m², or 11-100 j/m²). By way of example, the UV light source might typically operate at approximately 265 nm.

As an example, a container comprising a 2 milliwatt (mW) UVC LED may require an illumination area of no more than 60 cm² per second. Assuming an illumination cross section of approximately 12.5 cm² (to allow for 50% LED aging, internal cavity reflection losses, spectral efficiency, faster and deeper breathing etc.), assuming a UVC LED has wavelength of approximately 278 nm and a lifetime of approximately 10,000 hours (i.e. 3 years of use at 10 hours/day), and assuming human lung tidal capacity is approximately 0.5 liters at an average rate of approximately 15 breath/min, the required exposure volume may be approximately 125 cm³ per second. A calculation such as this may determine the overall volume of the container. Thus, in some embodiments, the container would be configured with an internal cavity (e.g. exposed) volume of approximately 125 cm³, at least approximately 125 cm³, or from approximately 80 cm³ to approximately 200 cm³ (or alternatively from approximately 80 cm³ to approximately 125 cm³). The container (including all of the electronic components) may be powered by batteries, such as 2 rechargeable AA batteries for approximately 30 hours (assuming 1% LED efficiency). A typical UVC LED may cost less than approximately $10 and its price would be expected to follow the Murphy law of decreasing 2 times every year or two. Thus the virus shield technology could be inexpensive enough for wide spread consumer use.

Disclosed mask embodiments typically would be configured to provide sufficient seal with a user's face (and typically would be formed of an air impermeable material) so that air into the mask must pass through the inlet(s) into the UV container. The mask would typically be configured to be worn on a user's face (e.g. covering the mouth and nose), such that air into the interior of mask must flow through the UV container. Some embodiments of the mask might have an exhalation valve (e.g. a one-way valve), configured to seal to prevent air entry into the mask through the valve but to allow exhalation air to exit the mask through the valve (and typically the exhalation pressure needed to open the valve would be set at a comfortable level for user breathing). In some embodiments, the mask might also contain a filter (e.g. configured to filter dust, pollen, bacteria, fungus spores, chemicals, etc.). For example, such a filter could be located over the container (e.g. with the container located within a filter covering such as an exterior mask comprising filter material) and/or over the inlet(s) to the container. Regardless, the mask configuration would typically prevent excessive pressure drop of the sort that might make user breathing difficult/laborious (e.g. breathing resistance should be minimized and kept to a level comfortable for the user). For example, filtering facepiece respirator R_{filter} for typical mask embodiments might be at or below approximately 88.2 Pa (measured at 85 l/min constant airflow). For masks with more breathing resistance, the mask may use a fan or other such mechanism to draw air in without requiring laborious user breathing. In some embodiments, the exposed volume of the container might be limited in such a way as to minimize ionized radicals exiting the outlet (e.g. so that the user does not receive a significant level/density to pose a health risk). This may be a concern in some instances, since UV light may interact with particles to cause ionized radicals in some instances. Thus, the volume of the UV container might be the minimum amount needed to kill the viruses at an effective level for health (for example approximately 125 cm³, or from approximately 80 cm³ to approximately 200 cm³, or alternatively from approximately 80 cm³ to approximately 125 cm³). Minimizing the volume should limit the amount of particle interaction that could result in ionized radicals. Additionally or alternatively some embodiment might be configured so that the container and/or mask (e.g. in proximity to the outlet of the container) absorb and/or block/filter or otherwise eliminate ionized radicals to prevent them from entering the interior of the mask to interact with the user's breathing.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A face mask comprising:
a container configured to expose breathable air to ultraviolet radiation, the container comprising:
at least one inlet allowing air flow into the container;
at least one light source configured to produce ultraviolet radiation, wherein the ultraviolet radiation is configured to kill viruses and/or other pathogens within the air flow;
a plurality of internal surfaces configured to reflect the ultraviolet radiation; and
at least one outlet allowing air flow out of the container toward a user's nose and/or mouth.

2. The face mask of claim 1, wherein the container further comprises a shield located proximate to the at least one inlet, the shield configured to prevent ultraviolet radiation from exiting the container via the inlet while allowing air to flow into the container via the inlet.

3. The face mask of claim 2, wherein the container further comprises at least one light sensor, wherein the at least one light sensor is configured to shut down the power for the UV LEDs when non-UV light is detected within the container.

4. The face mask of claim 2, wherein the container further comprises a shield located proximate to the at least one outlet, the shield configured to prevent ultraviolet radiation from exiting the container via the outlet while allowing air to flow out of the container via the outlet.

5. The face mask of claim 1, further comprising one or more of a mouth piece, a face piece, a face shell, a nose piece, or another similar element shaped to fit onto a portion of the user's face and configured to convey air from the outlet of the container to the nose and/or mouth of a user.

6. The face mask of claim 1, wherein the at least one light sources comprises one or more ultraviolet light-emitting diodes (UV LEDs).

7. The face mask of claim 1, wherein the at least one inlet comprises two or more inlets that are symmetrically spaced along a first side of the container.

8. The face mask of claim 7, wherein the outlet comprises at least one outlet that is symmetrically spaced along a second side of the container from the two or more inlets.

9. The face mask of claim 5, wherein the container is shaped to fit against a portion of the user's face, and the mask is configured to substantially seal against the user's face.

10. A container configured to expose breathable air to ultraviolet radiation, the container comprising:
at least one inlet allowing air flow into the container;
at least one light source configured to produce ultraviolet radiation, wherein the ultraviolet radiation is configured to kill viruses within the air flow;
a plurality of internal surfaces configured to reflect the ultraviolet radiation; and
at least one outlet allowing air flow out of the container toward a user's nose and/or mouth.

11. The container of claim 10, wherein the container further comprises a shield located proximate to the at least one inlet, the shield configured to prevent ultraviolet radiation from exiting the container via the inlet while allowing air to flow into the container via the inlet.

12. The container of claim 10, wherein the container further comprises a shield located proximate to the at least one outlet, the shield configured to prevent ultraviolet radiation from exiting the container via the outlet while allowing air to flow out of the container via the outlet.

13. The container of claim 10, wherein the ultraviolet radiation comprises from approximately 11 j/m² to approximately 33 j/m², and wherein the at least one light sources comprises one or more ultraviolet band C light-emitting diodes (UVC LEDs).

14. The container of claim 10, wherein the container comprises a volume of approximately 125 cm³.

15. The container of claim 10, further comprising at least one light sensor, wherein the at least one light sensor is configured to shut down the power for the UV LEDs when non-UV light is detected within the container

16. A method for preventing viruses from reaching a user's mouth and/or nose while wearing a mask, the method comprising:
directing breathable air into a container via one or more inlets;
emitting ultraviolet radiation within the container;
killing viruses contained in the air within the container via the UV radiation; and
directing the air out of the container via one or more outlets toward the user's mouth and/or nose.

17. The method of claim 16, further comprising reflecting the ultraviolet radiation by the inner surfaces of the container.

18. The method of claim 16, wherein the one or more inlets each comprise a shield configured to allow airflow into the corresponding inlet while preventing UV radiation from exiting the corresponding inlet, the method further comprising allowing air into the one or more inlets while preventing UV radiation from exiting out the one or more inlets.

19. The method of claim 16, wherein the one or more outlets each comprise a shield configured to allow airflow out of the corresponding outlet while preventing UV radiation from exiting the corresponding outlet, the method further comprising allowing air out of the container through the one or more outlets while preventing UV radiation from exiting out the one or more outlets into an interior of the mask.

20. The method of claim 16, further comprising detecting light in the container and, responsive to detecting light which is not UV, stopping emitting of UV radiation within the container.
